(19) United States
(12) Patent Application Publication
(10) Pub. No.: US 2024/0115127 A1
(43) Pub. Date: Apr. 11, 2024

(54) VARIABLE SCAN DEPTH SD-OCT SYSTEM AND VARIABLE CONFIGURATION OF OPTICAL FIBER SOURCES TO INCREASE THE EFFECTIVE SCAN RATE

(71) Applicant: Carl Zeiss Meditec, Inc., Dublin, CA (US)

(72) Inventors: Muzammil A. Arain, Milpitas, CA (US); Sophie Kubach, Menlo Park, CA (US); Jochen Straub, Dublin, CA (US); Tilman Schmoll, Vienna (AT)

(73) Assignee: Carl Zeiss Meditec, Inc., Dublin, CA (US)

(21) Appl. No.: 18/374,935

(22) Filed: Sep. 29, 2023

Related U.S. Application Data
(60) Provisional application No. 63/411,244, filed on Sep. 29, 2022.

(51) Int. Cl. A61B 3/10

(52) U.S. Cl. CPC A61B 3/102

(57) ABSTRACT

The system, method and device includes an optical coherence tomography (OCT) system switchable between single scan mode or multi-scan mode. This is achieved by use of multiple beam splitters to produce multiple OCT beams, but the system still uses a single light source, a single sample arm, and single reference arm to achieve a compact, more cost effective, system. Additionally presented is a spectral domain OCT system with a spectrometer having multiple grating, each providing a different depth and/or resolution imaging capability.

### CROSS-REFERENCE TO RELATED APPLICATIONS

This application claims priority to and the benefit of U.S. Provisional Patent Application No. 63/411,244 filed Sep. 29, 2022 and entitled “VARIABLE SCAN DEPTH SD-OCT SYSTEM AND VARIABLE CONFIGURATION OF OPTICAL FIBER SOURCES TO INCREASE THE EFFECTIVE SCAN RATE,” which is incorporated in its entirety for all purposes.

### FIELD

The present disclosure is generally directed to the field of Optical Coherence Tomography (OCT). More specifically, it is directed to increasing the scan rate of an OCT system. It is further directed to a method of changing the scan depth in a spectral domain OCT system (SD-OCT).

### BACKGROUND

Increasing the scan rate of an optical coherence tomography, OCT, system is desired to support wider scans without compromising spatial resolution. Some prior attempts at increasing the scan rate are known in the art. For example, publication: “High-power wavelength-swept laser in Littman telescope-less polygon filter and dual-amplifier configuration for multichannel optical coherence tomography” by Michael K. K. Leung et al. (Opt Lett. 2009 Sep 15; 34(18):2814-6. doi: 10.1364/0L.34.002814. PMID: 19756114) describes a multi-channel OCT prototype consisting of 12 bare ribbon fan out pigtails, where only the center 6 fibers are used as illumination source, each corresponding to a laser sweep generated by one facet of the polygon. All 6 channels are focused on the same scan depth, each imaging a separate strip of a sample, and each paired having a separate reference mirror. A second example is limited to 4 scanning beams to quadruple the OCT line rate, as described in a publication by Wolfgang Wieser et al. entitled “Multi-Megahertz OCT: High quality 3D imaging at 20 million A-scans and 4.5 GVoxels per second,” Opt. Express 18, 14685-14704 (2010). These two approaches do not mention any other fiber arrangements, neither do they offer the flexibility to select between a single, dual or quadruple scan beams. A third approach described in U.S. Pat. No. 9,164,240 (Optical buffering methods, apparatus, and systems for increasing the repetition rate of tunable light sources) uses a long spool of fiber to introduce a delay between 2 consecutive laser sweeps and is limited to doubling the A-scan rate.

Another difficulty associated with some OCT systems is that there is no simple way to adjust the scan depth of spectrum domain OCT (SD-OCT) systems. Typically, an SD-OCT system is designed and constructed for a desired/target scan depth, and once constructed, SD-OCT's scan depth cannot be altered. This limits the application of SD-OCTs as compared with other OCT architectures, such as swept source OCT (SS-OCT) whose scan depth can be made adjustable.

In various embodiments, the method/system may increase the effective scan rate of an OCT system.

In various embodiments, the scan rate of an OCT system may be variable.

In various embodiments, the method/system may alter the scan depth of an OCT system, and particularly the scan depth of an SD-OCT.

### SUMMARY

In various embodiments, the method/system may vary the scan speed of an OCT system by selectively scanning two or more sectors (e.g., concurrently or simultaneously scanning multiple sectors) of a test object (e.g., retina or other ophthalmic tissue) to define an image of larger field-of-view (FOV) with all sectors having a resolution similar to that of an OCT system capable to scanning a single sector at a time.

In various embodiments, an SD-OCT multiple gratings (with different grating period) can be slid/positioned in and out in the optical path to enable a variable scan depth SD-OCT.

In various embodiments, an optical coherence tomography (OCT) device may include: a light source for generating a beam of light; a first set of beam dividers and a second set of beam dividers, at least the second set of beam dividers having multiple beam dividers; an optical switch for selectively transferring the beam of light to one of the first set of beam dividers and the second set of beam dividers, where the first set of beam dividers directs a first portion of its received light into a reference arm and a second portion of its received light into a sample arm, the second set of beam dividers directs a first portion of its received light into said reference arm and a second portion of its received light into said sample arm; optics for directing the light in the sample arm to one or more locations on a sample; one or more detectors for receiving light returning from sample arm and the reference arm, and generating signals in response thereto; and a processor for converting the signals into image data.

In various embodiments, the second set of beam dividers provides multiple respective OCT beams, each directed to a different part of the sample. The multiple OCT beams may each scans a different part of the sample that, together, make up a composite image of the sample.

In various embodiments, the first set of beam dividers may include a single beam divider effective for generating a single scan beam, and the and multiple beam dividers of the second set of beam dividers each generates a respective separate scan beam.

In various embodiments, the optical switch switches between a single scanning mode and a multi-scanning mode. Additionally, the outputs of the first and second sets of beam dividers share a scanner.

In various embodiments, the respective light of the first and second sets of beams dividers returning from sample arm interfere with the same light returning from the reference arm.

The outputs of the first and second sets of beam dividers may be coupled to a respective fiber of a multi-fiber ferrule. In this case, the multi-fiber ferrule produces a respective OCT beam for each signal received at its respective fibers, and the OCT beams share the same optical path to the sample in the sample arm. In this case, the fibers of the multi-fiber ferrule are arranged to provide at least two scan beams covering the same area on the sample with a fixed delay delta-time defined as the number of A-scans contained in the distance between the two OCT beams. Also, the optical switch is a 1×N switch, the multi-fiber ferrule has N+1 fibers, the switch selects between one or multiples of two beam dividers, and each of the selected divider produces a separate OCT beam to scan a different area of the sample.

In various embodiments, a spectral domain optical coherence tomography (OCT) system may include: a broad light source for generating a beam of light; a beam divider for directing a first portion of the light into a reference arm and a second portion of the light into a sample arm; optics for directing the light in the sample arm to one or more locations on a sample; a spectrometer for measuring light returning from the sample and reference arms as a function of wavelength and generating signals in response thereto, where the spectrometer includes multiple different gratings for separately receiving the returning light; and a processor for converting the signals into image data. In this embodiment, different imaging depths and resolution are provided by the plurality of different gratings.

In various embodiments, the returning light is selectively and separately applied to a different one of said plurality of different gratings.

In various embodiments, the multiple, different gratings are movable, and a separate one of the multiple gratings is selectively moved into, and out of, the optical path of the returning light.

Also, the multiple gratings may be arranged to provide no overlap, and the returning light is applied to each of the multiple gratings, each generating a separate signals in response thereto.

A fuller understanding of the disclosure will become apparent and appreciated by referring to the following description and claims taken in conjunction with the accompanying drawings.

Several publications may be cited or referred to herein to facilitate the understanding of the present disclosure. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1 illustrates an example with an optical switch, in accordance with various embodiments.

FIG. 2 shows a Ferrule with three fibers, in accordance with various embodiments.

FIG. 3 shows OCT chief rays at start and end of a scan, where the OCT beams share the same galvo, in accordance with various embodiments.

FIG. 4 shows a free space optical switch, where the position of a reflector determines whether an OCT beam is concurrently transferred to Fiber Delivery 1 (FD1) and Fiber Delivery 3 (FD3), or is singularly transferred to Fiber Delivery 2 (FD2), in accordance with various embodiments.

FIG. 5 shows a bundle of 5 fibers, which provides the flexibility for a single scan on axis or 4 simultaneous scans off-axis, each covering a specific angular field of view (FOV) , in accordance with various embodiments.

FIG. 6 shows a top and side view of the bundle of fibers of FIG. 5, with 4 scanning beams and corresponding quadrants covered/scanned on the retina, in accordance with various embodiments.

FIG. 7 shows a typical setup of an SD-OCT system, in accordance with various embodiments.

FIG. 8 illustrates the concept of multiple gratings (with different grating period) that can be slid/positioned in and out in the optical path to enable a variable scan depth SD-OCT, in accordance with various embodiments.

FIG. 9 shows an example of a reflective spectrometer, in accordance with various embodiments.

FIG. 10 illustrates the concept of simultaneous acquisition of multiple scan depth data, in accordance with various embodiments.

FIGS. 11 and 12 illustrate an input system suitable for the configuration of FIG. 10, in accordance with various embodiments.

FIG. 13 illustrates a generalized frequency domain optical coherence tomography system used to collect 3D image data of the eye suitable, in accordance with various embodiments.

FIG. 14 shows an exemplary OCT B-scan image of a normal retina of a human eye, and illustratively identifies various canonical retinal layers and boundaries, in accordance with various embodiments.

FIG. 15 shows an example of an en face vasculature image, in accordance with various embodiments.

FIG. 16 shows an exemplary B-scan of a vasculature (OCTA) image, in accordance with various embodiments.

FIG. 17 illustrates an example computer system (or computing device or computer), in accordance with various embodiments.

### DETAILED DESCRIPTION

Increasing the scan rate of an optical coherence tomography (OCT) system is desired to support wider scans (e.g., larger field-of view, FOV) without compromising spatial resolution. The present approach is based on different configurations of pigtailed fibers, with each fiber delivering its own scan beam covering a portion of an image, which effectively increasing the scan rate. A characteristic of some embodiments is the inclusion of three fiber pigtails channels with a 1×2 fiber optic switch for single or dual scan beams for OCT imaging.

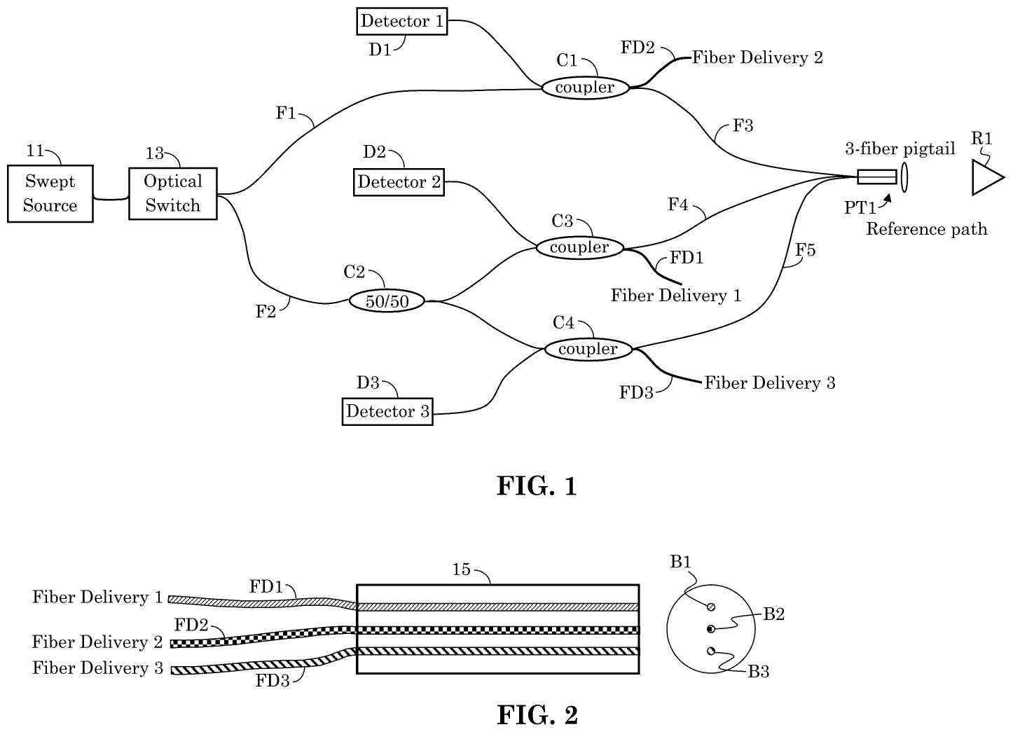

With reference to FIG. 1, a single light source (e.g., swept source) 11 is connected to an optical switch 13 that selectively couples light source 11 to one of two fibers (e.g., light paths) f1 and f2. Fiber f1 may be used for single scan mode. For example, optical switch 13 may couple a light signal from light source 11 to coupler C1 (e.g., beam divider), which transfers the light signal to sample fiber/line Fiber Delivery 2, FD2, and to fiber f3. Coupler C1 may be a 1- to-1 (1/1) coupler so that a copy of the light signal on fiber f1 is transferred to Fiber Delivery 2 and fiber f3. Fiber Delivery 2 would lead to a sample to be imaged and provides a single scan beam of an OCT system. A more detailed discussion of OCT systems is provided below. Fiber f3 leads to a reference arm (e.g., Reference path), which in the present example includes 3-fiber pigtail PT1 and reflector R1. As is discussed more fully below, the effective length of the light path in the reference path can be used to establish a scan depth position of a scan beam in the sample arm (or scan channel) of Fiber Deliver 2. The reference signal returning from the reference arm and the sample signal returning from the sample arm (e.g. along Fiber Delivery 2) are combined (e.g., interfere with each other) in coupler C1 and transferred to detector D1, where the signal is captured, processed, and optionally displayed on a monitor (not shown).

Fiber f2 may be used for dual scan mode. For example, optical switch 13 may couple light source 11 to a 50/50 coupler C1, which transfers the signal from fiber f2 to couplers C3 and C4 (e.g., half strength). Coupler C3 transfers the signal to fiber/line Fiber Delivery 1, FD1, which provides a first sample signal in the dual scan mode, and to fiber f4, which goes to the same reference arm (reference path) used in the single scan mode described above. The returning sample signal on Fiber Delivery 1 and the returning reference signal on fiber f4 are combined in coupler C3, and their combined signal is transferred to detector D2 for detecting/capturing and processing, as discussed below. Similarly, coupler C4 transfers the signal from 50/50 coupler C2 to Fiber Delivery 3, FD3, which provides a second sample signal in the dual scan mode, and to fiber f5, which goes to the same reference arm (reference path) used in the single scan mode described above. The returning sample signal on Fiber Delivery 3 and the returning reference signal on fiber f5 are combined in coupler C4, and their combined signal is transferred to detector D3 for detecting/capturing and processing. The two images produced from detectors D2 and D3 may be combined to generate a composite image in the present dual scan mode.

In addition to sharing the same reference path, the single and dual scan modes may also share the same sample arm (sample path). For example, with reference to FIG. 2, fiber delivery lines FD1, FD2, and FD3 may be coupled to a ferrule 15 to produce corresponding light beams B1, B2, and B3 (e.g., OCT beam/scan beam/scanning beam/probe beam), which may then be transferred to a scanner in the sample path (sample arm).

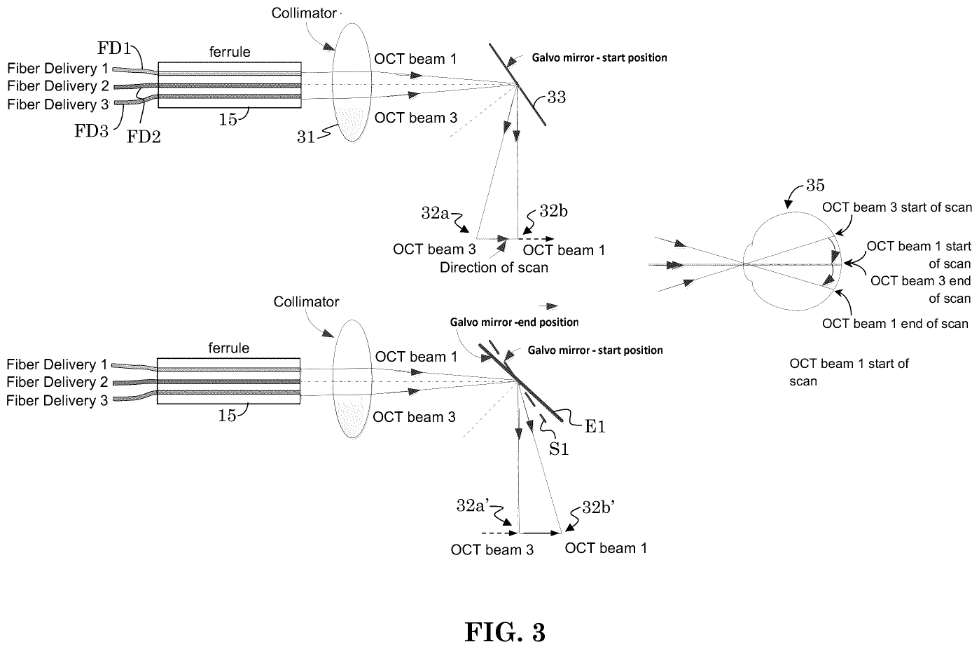

FIG. 3 provides an example of fiber delivery lines FD, FD2, and FD3 and ferrule 15 of FIG. 2 using the same sample path (or sample arm) for both single and dual scanning of an eye. For simplicity, some typical components of a sample arm/path are not shown in FIG. 3, but a more detailed discussion of the sample arm of an OCT system is provided below. As it would be understood, in single scan mode, Fiber Delivery 2 (FD2), which is here shown at the center of ferrule 15, would (optionally) pass through a collimator lens 31 to a scanning mechanism (e.g., galvo mirror 33), through an optical train, which may include a scan lens and an ophthalmic lens (not shown) to scan an eye 35. Again for simplicity, FIG. 3 illustrates an example scanning pattern for dual scan operation. Fiber deliveries FD1 and FD3 are shown offset from each other in ferrule 15 (e.g., on opposite sides of center fiber delivery FD2, in a linear arrangement) so as to define two offset starting scan positions 32a and 32b when scanner 33 is at its starting position S1. More specifically, fiber delivery lines FD1 and FD3 output corresponding OCT beam 1 and OCT beam 2, which are focused onto scanner 33 by collimator lens 31. As scanner 33 moves/tilts from its starting position S1 to its end position E1, OCT beam 3 is swept/scanned along a predefined direction of scan to its end position 32a′ (e.g., on the eye 35), which may coincide with the starting position of 32b OCT beam 1. Similarly, as scanner moves from its starting position to its end position, OCT beam 1 is scanned from its starting position 32b to its end position 32b′ on the eye 35. In the present example, the combined scanning path of OCT beam 1 and OCT beam 3 comprise the full scan length on eye 35.

The present embodiment provides a means to select between a single or dual scanning beam configuration from a three fiber ferrule. All three fibers share the same illumination source.

As discussed above, the three OCT beams (provide by fiber delivery lines FD1, FD2, and FD3) share the same scanner and optical elements contained in the OCT path (see FIG. 3). The three fibers FD1, FD2, and FD3 are arranged such that the central fiber FD2 is aligned to the optical axis and the other two fibers (FD1 and FD3) are separated by half the retinal (e.g., scan) field-of-view that is to be image divided by the system magnification (see FIGS. 2 and 3).

The light scatted from the (eye) tissue and coupled into the three fibers FD1, FD2 and FD3 interferes with the same reference beam (shown in FIG. 1). Detection of interference may use three photodiode detectors D1, D2 and D3, one for each fiber delivery. Fiber optical switch 13 is employed for selecting between a dual scan and a single scan beam configuration.

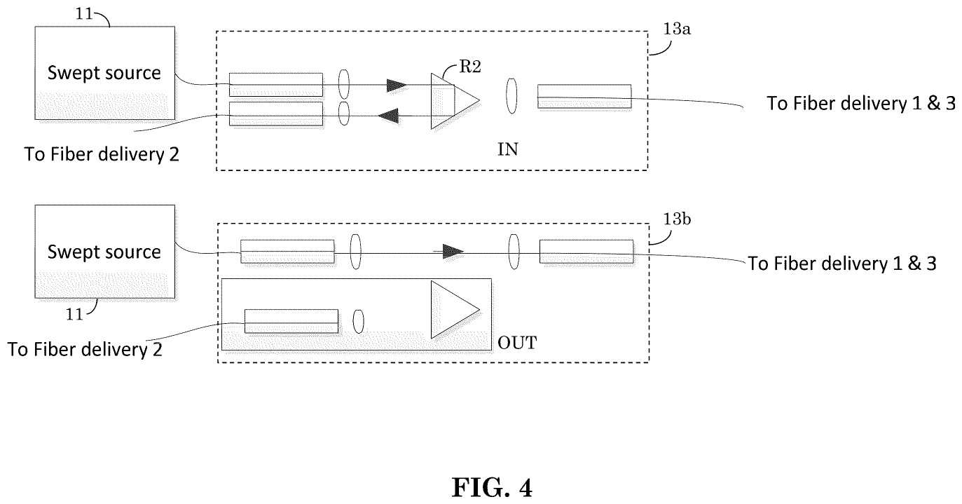

An exemplary free space optical switch suitable for use as optical switch 13 is illustrated in FIG. 4. All elements similar to those of FIG. 1 have similar reference characters and are described above. In the present embodiment, optical switch 13 of FIG. 1 may be placed in one of two positions 13a and 13b (e.g., operating modes). In a first position 13a, a reflector R2 blocks a light path to Fiber Delivery 1 (FD1) and Fiber Delivery 3 (FD3), and instead reflects light from swept source 11 to Fiber Delivery 2 (FD2). In this first position 13a, no light is transferred to fibers FD1 and FD3. In a second position 13b, reflector R2 is moved out of the light path from swept source 11 to fibers FD1 and FD3. In this manner, light from swept source 11 is transferred to Fiber Delivery 1 and Fiber Delivery 3, and no light is transferred to Fiber Delivery 2. Thus, an OCT beam is either delivered to fibers FD1 and FD3, or delivered to fiber FD2.

Unlike the prior art, the present embodiment provides the flexibility to switch between a single scan and a dual scan, OCT scan beam. In the present embodiment, the three channels (e.g., OCT channels) share the same illumination source, same sample path, and the same reference path, thus reducing cost and simplifying its implementation. Another novel characteristic of some of the present embodiments is that they provide the flexibility to deliver single, dual, or quadruple scanning beams using a fiber optic switch and a bundle of five fibers covering from one to two to four quadrants on a retina. Thus, in embodiments, the scan rate proportionally increases with the number of quadrants being covered (e.g., being concurrently/simultaneously scanned). Advantageously, the embodiments can be expanded to provide the flexibility to deliver N scanning beams via and a bundle of N fibers covering from one to N quadrants on a retina, and effectively increasing the scan rate by up to a factor of N.

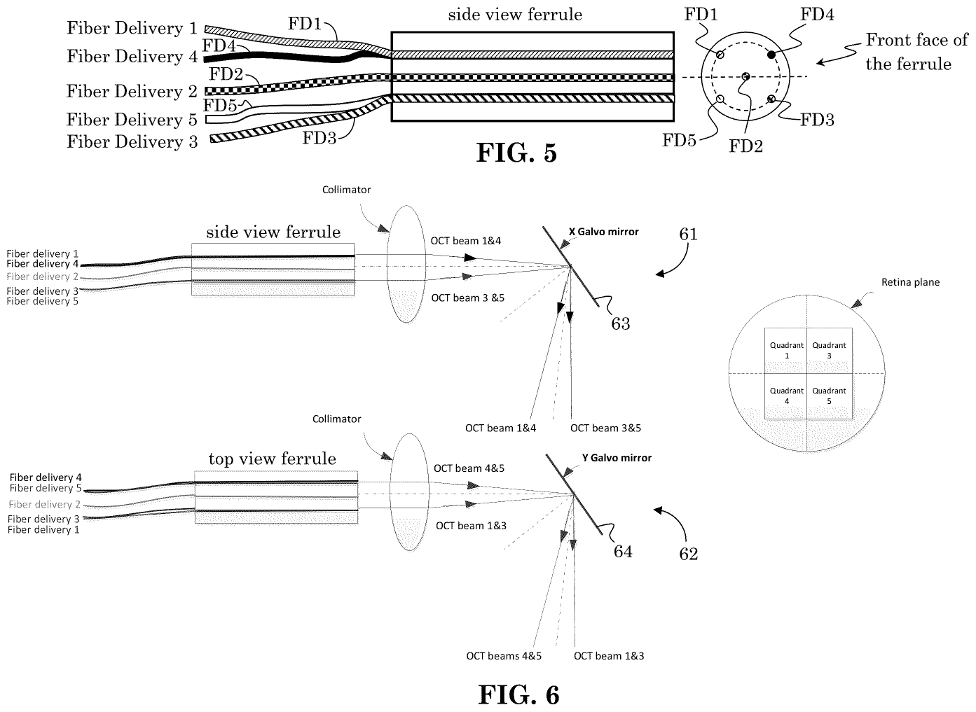

Thus, in various embodiments, the system may optionally be expanded to multi-scan beams from a bundle of fibers. For example, five fibers sources, may be arranged with one on axis and the other four arranged at a fixed distance from the optical axis such as to provide simultaneous coverage of four quadrants of the retina. FIG. 5 illustrates a bundle of five fibers (FD1-FD5) that provides the flexibility for a single scan on axis or multiple (e.g., two or four) simultaneous scans off-axis, each covering a specific angular FOV. FIG. 5 provides a side view of a ferrule with five fibers suitable for switching between single scan mode, dual scan mode, and quadruple scan mode. All elements similar to those of FIGS. 1-4 have similar reference characters and are discussed above. As before, one fiber (e.g., Fiber Delivery 2, FD2) is aligned along the axis used for single scan mode. Two other fibers, Fiber Delivery FD1 and FD may be used for dual scan operation, as described above, and two additional fibers, Fiber Delivery FD4 and FD5, provide two additional quadrant scanning to achieve quad scan mode. As before, fibers FD1 and FD 3 maybe be on opposite sides of, and aligned with, axially positioned fiber FD2, and an end scan position of FD1 may coincide with the starting scan position of FD3. Similarly, fibers FD4 and FD5 may be on opposite sides of, and aligned with, axially positioned fiber FD2, and an end scan position of FD4 may coincide with the starting scan position of FD5. As discussed below, in order for fibers FD1, FD3, FD4, and FD5 to comprise four contiguous scanning quadrants, the end scan position (or end scan line) of one pair of fibers (e.g., FD1 and FD3) may coincide with the start scan position (or start scan line) of the other pair of fibers (e.g., FD4 and FD5).

FIG. 6 illustrates a scanning arrangement for simultaneously scanning four quadrants of a retinal plane (e.g., the back of eye). FIG. 6 provides a side view 61 and a top view 62 of the bundle of fiber with four scanning beams and the corresponding quadrants covered on the retina plane (e.g., eye). An X-galvo mirror 63 is arranged so that the starting scan positions of OCT beams 1 and 4 are offset along the X-axis from the starting scan position of OCT beams 3 and 5, and the end X-scan position of OCT beams 1 and 4 correspond to the starting X-scan positions of OCT beams 3 and 5 in a manner similar to that of FIG. 3. A Y-galvo mirror 64 is arranged so that the OCT beams 4 and 5 and OCT beams 1 and 3 form a continuous scan flow along the Y-scan direction.

The above method of increasing the effective scan rate may be applied to multiple OCT types, such as swept-source OCT (SS-OCT), spectral domain OCT (SD-OCT), and time domain OCT (TD-OCT), but differences in the different type of OCT architectures complicate the implementation of other features. For example, there are multiple ways that scan depth can be changed in SS-OCT, but heretofore, there has not been an easy way to change the scan depth in SD-OCT. For example, a method for varying the imaging depth in Fourier domain optical coherence tomography (e.g., for an SS-OCT) is described in US 20130120757, herein incorporated in its entirety by reference, but such a method is not applicable to SD-OCT systems. Usually altering the scan depth of an SD-OCT system includes changing or replacing its (light) source or redesigning its spectrometer. This restricts the combination of scan depth, resolution, and speed to only one value. Herein is proposed a method of resolving this limitation by using multiple gratings. The variable scan depth will allow SD-OCT to be used in multiple use cases. Previously, SD-OCT has been limited to using one scan speed which was usually set by designing the spectrometer in such a way that the detector was used at its maximum speed and best resolution. There could be use cases where a lower resolution may be acceptable but with even higher speed. The embodiment presented below provides multiple options for selecting speed/resolution combinations.

In various embodiments, the systems, methods and applications are for adjusting the imaging depth of a spectral domain optical coherence tomography (SD-OCT) system via the use of multiple gratings inside a single spectrometer without the need of changing the spectral source. The proposed technique can provide sequential as well as parallel acquisition of multiple scan depth information. Some advantages of the present disclosure include, for example:
Variable scan depth SD-OCT without the need to change the source spectral properties;
Option of multiple scan depths at the same time using one or more multi-line detector; and
Low resolution, but high speed data acquisition.

### General OCT Systems for Ophthalmology

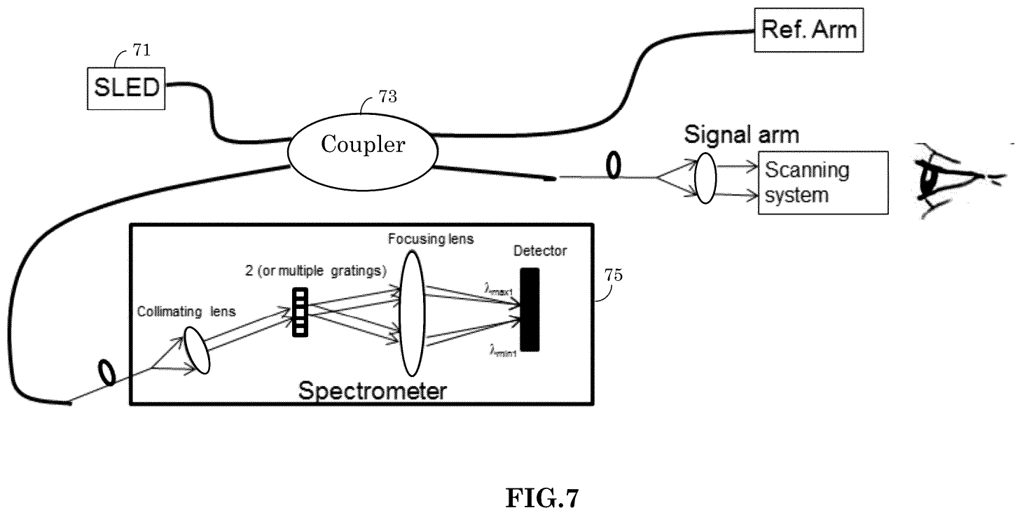

FIG. 7 shows a typical setup of an SD-OCT system, in various embodiments, such as used is ophthalmological instruments. Here a broadband (light) source 71 usually an SLED (Superluminescent Light Emitting Diodes) is used in an interferometric set-up. The signal arm (or sample arm) is directed towards the patient's eye where a scanning system scans the light into the patient's eye. The return signal is mixed with light returning from the reference arm interferometrically inside the coupler 73 and the mixed signal is sent to a spectrometer 75 where an CCD (charge-coupled device) or CMOS (complementary metal-oxide semiconductor) based (e.g., line-scan) sensor (e.g., photosensor or camera) is used to extract 3-D (3 dimensional) information of the patient's eye.

This scan depth zmax of the system is determined by Eq. 1.

$${z}_{max} = \frac{N {λ}_{0}^{2}}{4 {λ}_{full}} \qquad ( 1 )$$

where N is the total number of pixels in the line-scan camera, λₒ is the center wavelength of the source, λ_{full} is preferably ≥2.26618Δλ to fulfill the Nyquist limit criterion for a source of FWHM (Full width at half maximum) spectral width of Δλ.

From Eq. 1 it can be seen that for a given spectral source width λ_{full}, if the spectrum is spread over more pixels, the scan depth will increase. This may include the use of a camera with more pixels. Optically, the total of pixels that will see the full spread λ_{full}, is a function of the focusing lens (focal length) and the grating period used in the spectrometer.

One way to change the value of N is to change the focal length of the lens and reposition the detector accordingly. This will be a cumbersome process. In this configuration, we propose the use of multiple gratings that can be inserted in the optical path of the beam to enable variable scan depth. Suppose a grating period of Γ₁ (lines per mm) spreads λ_{full} on N₁ number of pixels on the line scan camera, this results in a scan depth of zₘₐₓ₁. To double the scan depth to zₘₐₓ₂=2* zₘₐₓ₁, a grating with Γ₂₌₂*Γ₁ (lines per /mm) will spread the spectrum to 2* N₁ pixels on the line scan camera.

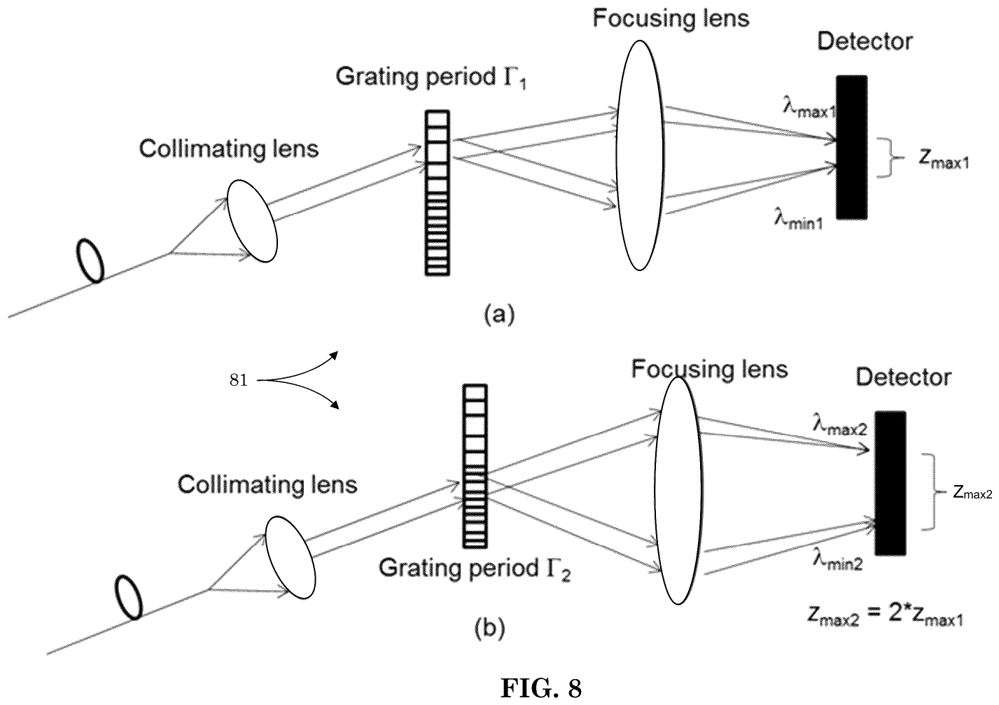

FIG. 8 illustrates this concept of multiple gratings (with different grating period) that can be slid/positioned in and out in the optical path to enable a variable scan depth SD-OCT. Part (a) of FIG. 8 shows a spectrometer 81 similar to that of FIG. 7, but having a grating period of Γ1 resulting in a scan depth Zmax1. Part be (b) shows the grating of part (a) shifted/slid/repositioned so that a grating period Γ₂ takes it place, which is here selected to be twice of Γ₁ to enable doubling of the scan depth to Zmax2.

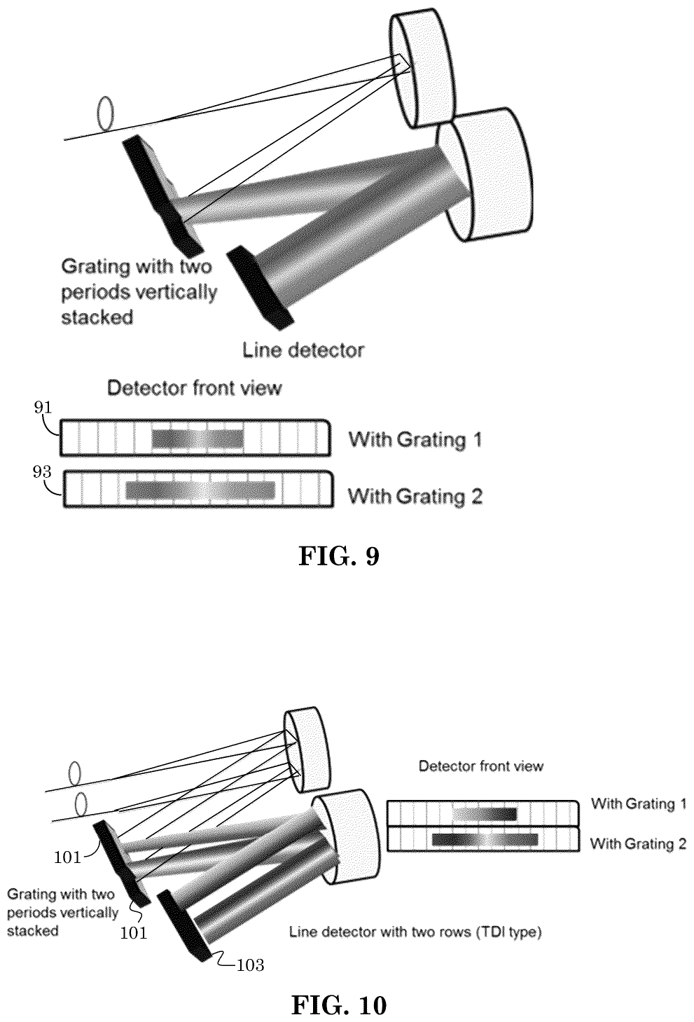

FIG. 8 further shows the concept of a transmissive spectrometer. The concept can be applied to a reflective spectrometer as well which may provide more options. FIG. 9 shows an example of reflective spectrometer.

FIG. 9 illustrates the concept of multiple gratings applied to reflective set up. That is multiple grating 91 and 93 (each with a different grating period as discussed above, are in the optical path to the line detector.

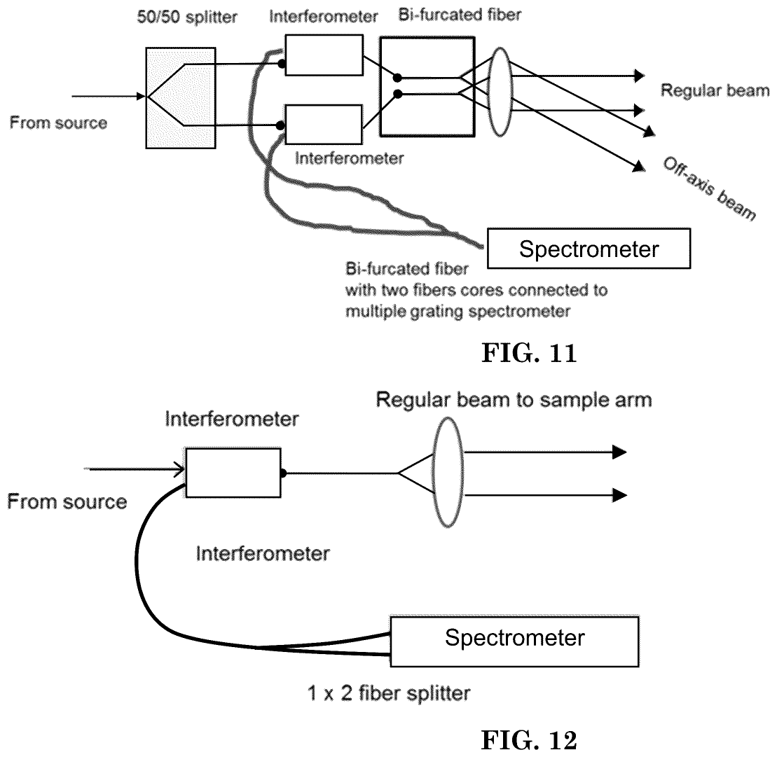

It can be seen that apart from sliding/repositioning the gratings in and out, one could potentially move the fiber and a mirror or enable coordinated movement on the two mirrors to enable a variable scan depth. Another configuration can be to use multiple fiber inputs stacked vertically. This configuration can provide simultaneous measurements of variable scan depths in an SD-OCT as shown in FIG. 10. That is, FIG. 10 illustrates the concept of simultaneous acquisition of multiple scan depth data. In the present approach, multiple gratings 101 and 102 are provided, each of which produces a separate image on the detector 103. For example, a two row detector, one per graining, may be used. It is noted that for the configuration of FIG. 10, one could employ multiple row line detectors which are common in TDI (time delay and integration imaging). The fibers can be arranged so that the two fibers are imaged on two separate lines. FIGS. 11 and 12 illustrate exemplary input systems to a spectrometer suitable for the present configuration(s).

FIG. 11 illustrates the use of two interferometers to transfer to mixed signals to the spectrometer. In the present example, a bi-furcating fiber with two fiber cores are connected to multiple gratings in the spectrometer. This illustrates the concept of using multiple fibers via multifurcated fibers to be used as the source system to the variable scan depth spectrometer, as discussed above. In both FIG. 11 and FIG. 12, for ease of discussion, the return signal from the reference arm is not shown (see FIG. 7), but as discussed above, the returning light from the reference arm and sample are mixed in one or more interferometer (e.g., coupler) and sent to the spectrometer. FIG. 12 illustrates the use of one interferometer, and the mixed signal is sent along a 1×2 fiber. Although only a 1×2 fiber is shown in the present example, the present concept can be extended to multiple fibers (e.g., greater than two fibers), where the signal from each fiber is directed to a respecting grating in the spectrometer, as discussed above. That is, FIG. 12 shows the use of 1×2 (or ×N) fiber coupler to enable the variable scan depth SD-OCT spectrometer of FIG. 10.

Exemplary benefits of various embodiments include:
1. A three fiber configuration that provides the flexibility to deliver a single or dual scanning beams for OCT imaging;
2. Dual simultaneous scanning beams double the scan rate.
3. Dual simultaneous scanning beams reduce the overall scan duration.
4. Optical switch to select a single fiber or a dual fiber delivery system.
5. Common scanner: Output beams from the three fibers share the same scanner.
6. Common reference path: Reflected light beam from the sample arm coupled back to the three fibers interfere with the returned light from the same reference beam.
7. Common light source: The three (or more) fiber optics share the same light source.
8. Bundle of optical fiber sources delivering a plurality of OCT beams sharing the same optical path to the eye leads to a compact design.
9. The fibers can be arranged such as to provide at least 2 scan beams covering the same area on the eye but with a fixed delay delta t given by the number of A-scan contained in the distance between the 2 scanning beams.
10. The above embodiment(s) can be expanded to provide the flexibility to deliver single, dual or quadruple scanning beams using a 1×N optical switch and a bundle of five fibers covering from 1 to 2 to 4 quadrants on retina. The scan rate proportionally increases with the number of quadrants/sectors/areas.
11. The above embodiment(s) can be expanded to provide the flexibility to deliver N scanning beams via and a bundle of N fibers covering from N quadrants/sectors/areas on a retina (sample), effectively increasing the scan rate up by a factor of N.

The present embodiments also provide for a spectral domain optical coherence tomography (SD-OCT) system generating images of an eye, or other sample, including: a light source for generating a probe beam wherein the light source is a broadband light source; optics for scanning the beam over a set of transverse locations across the eye; a spectrometer for measuring light returned from the eye as a function of wavelength that acquires data at a data acquisition rate; and a processor for generating images of the eye based on the output of the detector over the sampling of wavelengths, where the SD-OCT system switches imaging modes with different imaging depths and resolution by using multiple gratings, moving sequentially at each transverse location. Alternatively, the SD-OCT system may support multiple simultaneous imaging modes with different imaging depths and resolution by using multiple gratings, at each (or at different) transverse locations.

In this embodiment, multiple beams may be used at the input of the spectrometer through fiber splitter. Alternatively, multiple beams may be used at the input of the spectrometer through multi-furcated fiber used (e.g., to scan) at different transverse locations.

Additionally, a grating with multiple grating periods on a single substrate may be used. In embodiments, gratings with different gratings periods are stacked together on a moving mechanism.

Also, the grating period of a single grating can be tuned by applying a control signals, e.g., electronically tunable gratings.

In embodiments, a combination of fiber and/or mirror may be used to direct the light output to the detector.

Additionally, multiple beams may be used at the input of the spectrometer through a fiber splitter. In embodiments, the detector is a time delay and integration (TDI) line scan detector with multiple rows of output. Also, gratings with different gratings periods are stacked together.

Hereinafter is provided a description of various hardware and architectures suitable for various embodiments.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

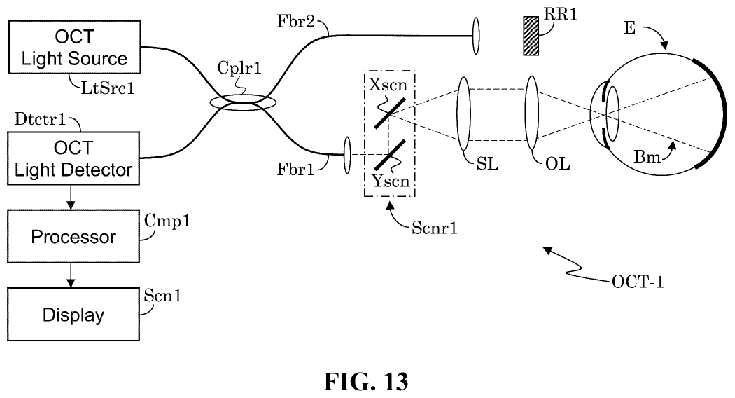

FIG. 13 illustrates a generalized frequency domain optical coherence tomography (FD-OCT) system used to collect 3D image data of the eye suitable for use in various embodiments. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produce substantially collimated light, and ophthalmic lens OL may then focus onto the sample. The present example illustrates a scan beam that should be scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be included to scan the line-field beam across the sample to span the desired FOV. If the scan beam were a full-field beam (e.g., a full-field OCT), no scanner may be needed, and the full-field light beam may be applied across the entire, desired FOV at once.

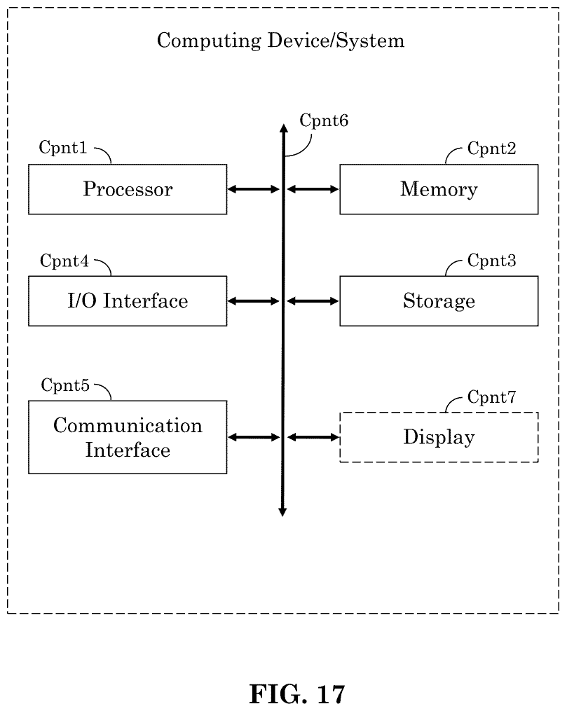

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retroreflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 17. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13):2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm may have a tunable optical delay to generate interference. Balanced detection systems are typically used in TD-OCT and SS-OCT systems, while spectrometers are used at the detection port for SD-OCT systems. Various embodiments could be applied to any type of OCT system. Various embodiments could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

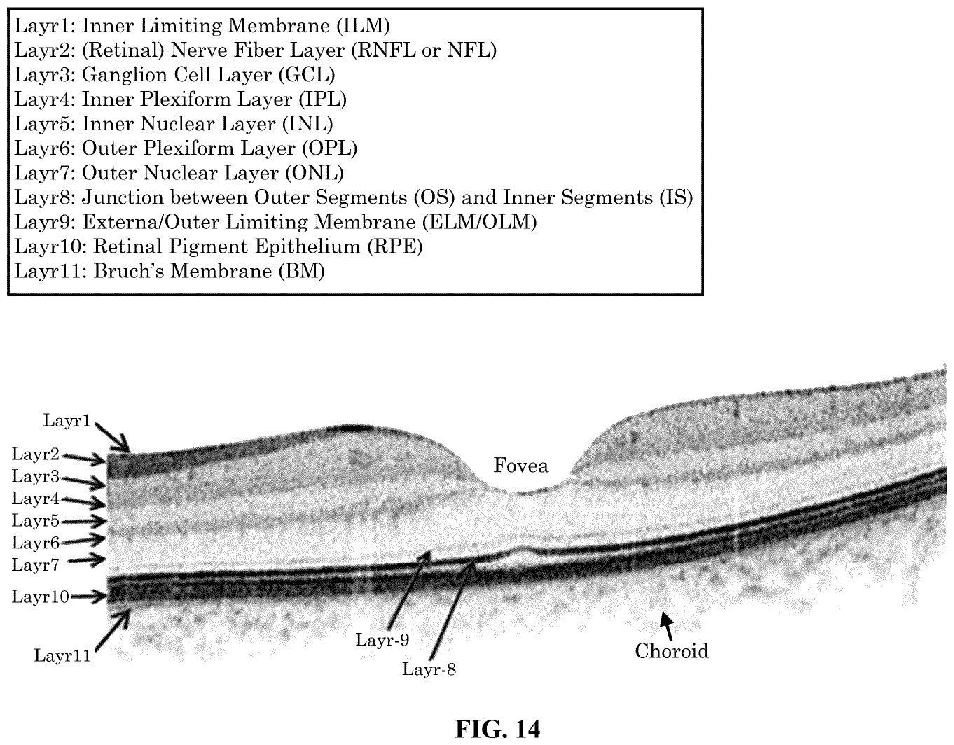

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 14. An OCT B-scan of the retinal provides a view of the structure of retinal tissue. For illustration purposes, FIG. 14 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

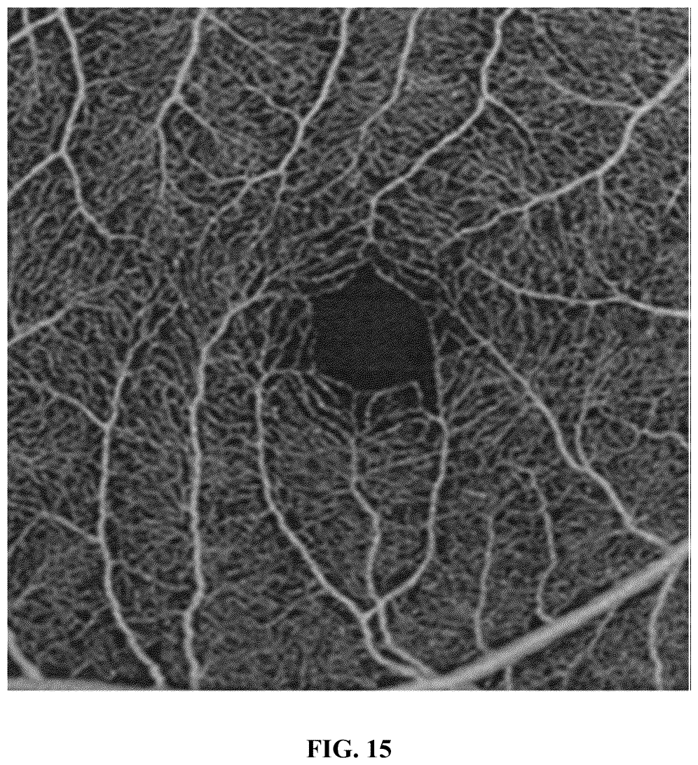

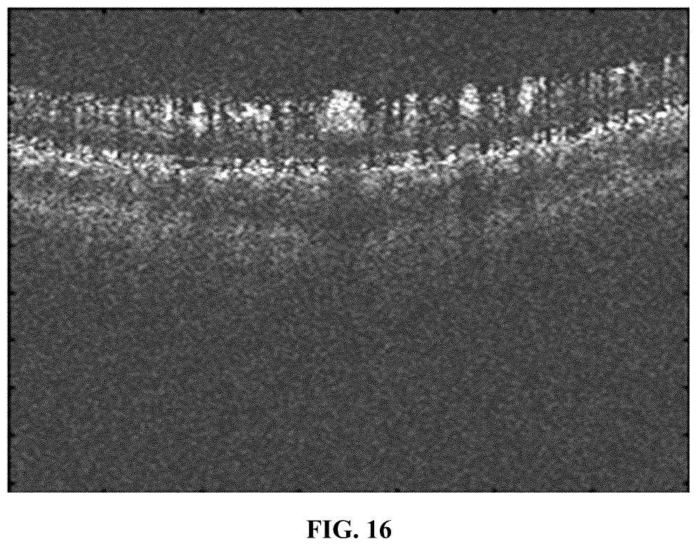

FIG. 15 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 16 shows an exemplary B-scan of a vasculature (OCTA) image. As illustrated, structural information may not be well-defined since blood flow may traverse multiple retinal layers making them less defined than in a structural OCT B-scan, as shown in FIG. #@D. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique including the injection of a dye to observe vascular flow in the retina. Additionally, in dry age-related macular degeneration, OCTA has been used to monitor a general decrease in choriocapillaris flow. Similarly in wet age-related macular degeneration, OCTA can provides a qualitative and quantitative analysis of choroidal neovascular membranes. OCTA has also been used to study vascular occlusions, e.g., evaluation of nonperfused areas and the integrity of superficial and deep plexus.

### Computing Device/System

FIG. 17 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6.The computer system may optionally also include a display Cpnt7, such as a computer monitor or screen.

Processor Cpnt1 includes hardware for executing instructions, such as those making up a computer program.For example, processor Cpnt1 may be a central processing unit (CPU) or a general-purpose computing on graphics processing unit (GPGPU).Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables.Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM).Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user).For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the disclosure is described in conjunction with several specific embodiments, it is evident to those skilled in the art that many further alternatives, modifications, and variations will be apparent in light of the foregoing description. Thus, the disclosure is intended to embrace all such alternatives, modifications, applications and variations as may fall within the spirit and scope of the appended claims.

We Claim:

1. An optical coherence tomography (OCT) device comprising:
a light source for generating a beam of light;
a first set of beam dividers and a second set of beam dividers, at least the second set of beam dividers including a plurality of beam dividers;
an optical switch for selectively transferring the beam of light to one of the first set of beam dividers and the second set of beam dividers, the first set of beam dividers directing a first portion of its received light into a reference arm and a second portion of its received light into a sample arm, the second set of beam dividers directing a first portion of its received light into said reference arm and a second portion of its received light into said sample arm;
optics for directing the light in the sample arm to one or more locations on a sample;
one or more detectors for receiving light returning from sample arm and the reference arm, and generating signals in response thereto; and
a processor for converting the signals into image data.

2. The device of claim 1, wherein the second set of beam dividers provides a respective plurality of OCT beams, each directed to a different part of the sample.

3. The device of claim 2, wherein the plurality of OCT beams each scans a different part of the sample that comprise a composite image of the sample.

4. The device of claim 1, wherein:
the first set of beam dividers includes a single beam divider effective for generating a single scan beam; and
the plurality of beam dividers of the second set of beam dividers each generate a respective separate scan beam.

5. The device of claim 1, wherein the optical switch switches between a single scanning mode and a multi-scanning mode.

6. The device of claim 1, wherein the outputs of the first and second sets of beam dividers share a scanner.

7. The device of claim 1, wherein the respective light of the first and second sets of beams dividers returning from sample arm interfere with the same light returning from the reference arm.

8. The device of claim 1, wherein the outputs of the first and second sets of beam dividers are coupled to a respective fiber of a multi-fiber ferrule, and the multi-fiber ferrule produces a respective OCT beam for each signal received at its respective fibers, the OCT beams share the same optical path to the sample in the sample arm.

9. The device of claim 8, wherein the fibers of the multi-fiber ferrule are arranged to provide at least two scan beams covering the same area on the sample with a fixed delay delta-time defined as the number of A-scans contained in the distance between the two OCT beams.

10. The device of claim 8, wherein the optical switch is a 1×N switch, the multi-fiber ferrule has N+1 fibers, the switch selects between one or multiples of two beam dividers, and each of the selected divider produces a separate OCT beam to scan a different area of the sample.

11. A spectral domain optical coherence tomography (OCT) system comprising:
a broad light source for generating a beam of light;
a beam divider for directing a first portion of the light into a reference arm and a second portion of the light into a sample arm;
optics for directing the light in the sample arm to one or more locations on a sample;
a spectrometer for measuring light returning from the sample and reference arms as a function of wavelength and generating signals in response thereto, the spectrometer including a plurality of different gratings for separately receiving the returning light; and
a processor for converting the signals into image data;
wherein different imaging depths and resolution are provided by the plurality of different gratings.

12. The system of claim 11, wherein the returning light is selectively and separately applied to a different one of said plurality of different gratings.

13. The system of claim 12, wherein the plurality of different gratings are movable, and a separate one of said plurality of different gratings is selectively moved into, and out of, the optical path of the returning light.

14. The system of claim 11, wherein the plurality of different gratings are arranged to provide no overlap, and the returning light is applied to each of the plurality of different gratings, each generating a separate signals in response thereto.
